(19) United States
(12) Patent Application Publication
(10) Pub. No.: US 2023/0201407 A1
(43) Pub. Date: Jun. 29, 2023

(54) CORONA STERILIZATION MODULE

(71) Applicant: WEIWEICANQIONG(SHANGHAI) HEALTH TECHNOLOGY CO, LTD., Shanghai (CN)

(72) Inventor: MING ZHANG, Shanghai (CN)

(21) Appl. No.: 17/964,901

(22) Filed: Oct. 12, 2022

Related U.S. Application Data
(63) Continuation of application No. PCT/CN2021/140962, filed on Dec. 23, 2021.

(30) Foreign Application Priority Data
Dec. 24, 2021 (CN) 2021233033514

(51) Int. Cl. A61L 9/18

(52) U.S. Cl. CPC A61L 9/18

(57) ABSTRACT

An ultra-thin corona sterilization module is related, including an insulation enclosure, a apertured negative electrode plate and a positive electrode holder, the apertured negative electrode plate is at the bottom of the insulation enclosure and has many mounting holes for mounting cylinder-shaped tubes. Each cylinder-shaped tube has a bottom air inlet fixed in the mounting hole, and a top opening as an air outlet. The positive electrode holder is grid-shaped, positioned at the top of the insulation enclosure and spaced apart from the air outlet, with an electrode needle suspended above axis center of each metal cylinder-shaped tube. The electrode needle's end is at a skeleton of the positive electrode holder. The electrode needle's tip is spaced 5-10 mm from the air inlet. The disclosure applies electrostatic catalytic technology to air sterilization by generating a corona field through high voltage electrostatic, thus achieving air sterilization in a manned environment.

### TECHNICAL FIELD

The present disclosure relates to a field of air sterilization and purification, and specifically to an ultra-thin corona sterilization module.

### BACKGROUND

Due to the impact of the Covid-19 epidemic, more and more attention is being paid to air sterilization, a current air sterilization way is to perform sterilization in an unmanned environment, by using ultraviolet light, spraying disinfectant liquid, etc., while it lacks a technical means of air sterilization in a manned environment at any time. Current ionization technology is mainly configured for dust removal and deodorization, usually at a voltage of more than 8000 volts, current products have a thickness of over 10 cm, the dust removal operation may cause electric field breakdown and lead to ozone exceeding, and thus involves problems such as ozone exceeding, crackling noise, product thickness, and difficulties of maintenance.

### SUMMARY

The present disclosure provides an ultra-thin corona sterilization module, which cleverly applies electrostatic catalytic technology to air sterilization, making full use of the sterilization function of corona field, and realizing air sterilization of the manned environment by generating corona field, which not only can eliminate ozone exceeding, but also involves no noise, easy maintenance, and safety for human body, equipment and environment.

According to a first aspect, an embodiment provides a corona sterilization module including an insulation enclosure, an apertured negative electrode plate and a positive electrode holder. The apertured negative electrode plate is positioned at a bottom of the insulation enclosure, the apertured negative electrode plate is provided with a number of mounting holes for a mounting metal cylinder-shaped tube, with a bottom air inlet of the metal cylinder-shaped tube fixed in the mounting holes of the apertured negative electrode plate, and a top opening of the metal cylinder-shaped tube is configured to be an air outlet. The positive electrode holder is grid-shaped, the positive electrode holder is positioned on a top of the insulated enclosure and spaced apart from the air outlet of the cylinder-shaped tube, with an electrode needle suspended on above of each cylinder-shaped tube, an end of the electrode needle being mounted at a skeleton of the positive electrode holder, and the electrode needle has a needle tip spaced apart from the air inlet of the cylinder-shaped tube. An external power connection case and a high voltage pack are provided in the insulation enclosure, the high voltage pack having a positive pole electrically connected to the positive electrode holder and a negative pole electrically connected to the apertured negative electrode plate.

In some embodiments, the apertured negative electrode plate and the positive electrode holder are fixedly connected to the insulation enclosure via insulated connecting posts, respectively.

In some embodiments, the apertured negative electrode plate is configured parallelly to the positive electrode holder.

In some embodiments, the needle tip of the electrode needle is spaced 5 mm-10 mm from the air inlet of the metal cylinder-shaped tube.

In some embodiments, the positive electrode holder is spaced more than 10 mm from the metal cylinder-shaped tube.

In some embodiments, the high voltage pack has a voltage of 4000v-8000v and a current of 200 μA-1 mA.

In some embodiments, the metal cylinder-shaped tube has a height of 1 cm-3.6 cm.

In some embodiments, the electrode needle is located in an axis-center position inside the metal cylinder-shaped tube.

In some embodiments, a lower area from the air inlet to a position 5 mm above the needle tip of the electrode needle is configured to be a corona zone and an upper area from the position 5 mm above the needle tip of the electrode needle to the air outlet is configured to be an adsorption zone.

In some embodiments, the corona zone is configured to be a rotating corona field with a mineralization effect on viruses.

Based on the above embodiments, the present disclosure utilizes high-voltage electrostatic catalytic technology to form a high-speed rotating corona field in an ionized area, thus achieving strong mineralized sterilization on the air passing through; the ultra-thin design of the present disclosure facilitates the sterilization equipment being adaptable to air conditioner in any style and has a more aesthetically pleasing appearance, and it can also be designed by a combination of multi-layer of the present sterilization module to form an enhanced sterilization equipment with an air-passing sterilization ability of more than 90% at one time.

### BRIEF DESCRIPTION OF DRAWINGS

FIG. 1 Illustrates a front view of an embodiment of an ultra-thin corona sterilization module.

FIG. 2 Illustrates a rear view of an embodiment of an ultra-thin corona sterilization module.

FIG. 3 Illustrates a side sectional view of an embodiment of an ultra-thin corona sterilization module.

FIG. 4 Illustrates a schematic diagram of a corona zone and an adsorption zone of one embodiment.

FIG. 5 Illustrates a three-dimensional view of an embodiment of an ultra-thin corona sterilization module.

FIG. 6 Illustrates a partial disassembly of an embodiment of an ultra-thin corona sterilization module.

### DETAILED DESCRIPTION OF EMBODIMENTS

The disclosure will be further described in detail below through specific implementations in conjunction with the accompanying drawings. Associated similar element reference numerals are used for similar elements in different implementations. In the following implementations, many details are described such that the disclosure may be better understood. However, it may be effortlessly appreciated by a person skilled in the art that some of the features may be omitted, or may be substituted by other elements, materials, and methods in different cases. In certain cases, some operations involved in the disclosure are not displayed or described in the specification, which is to prevent a core part of the disclosure from being obscured by too much description. Moreover, for a person skilled in the art, the detailed description of the involved operations is not necessary, and the involved operations can be thoroughly understood according to the description in the specification and general technical knowledge in the art.

In addition, the characteristics, operations, or features described in the specification may be combined in any appropriate manner to form various implementations. Meanwhile, the steps or actions in the method description may also be exchanged or adjusted in order in a way that is obvious to a person skilled in the art. Therefore, the various orders in the specification and the accompanying drawings are merely for the purpose of clear description of a certain embodiment and are not meant to be a necessary order unless it is otherwise stated that a certain order must be followed.

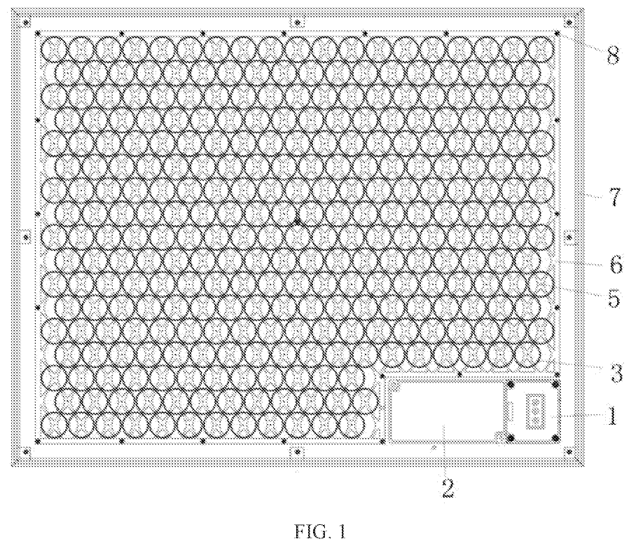

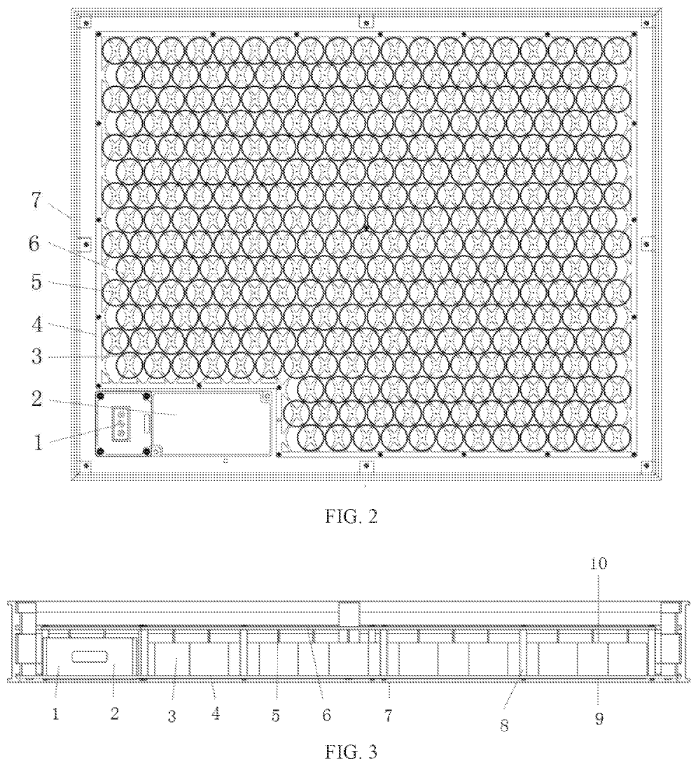

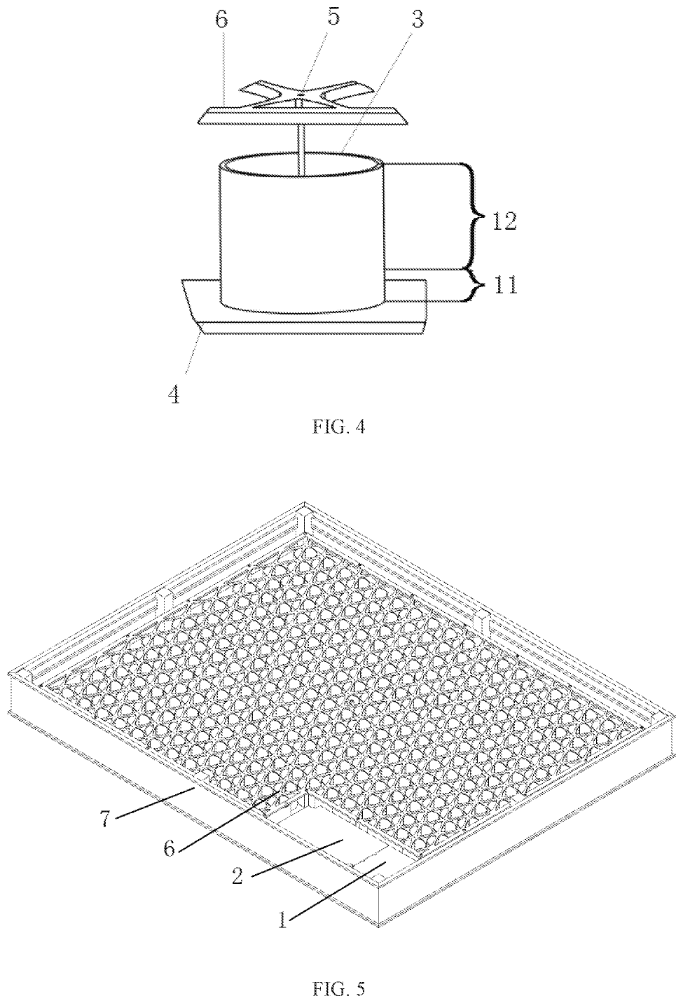

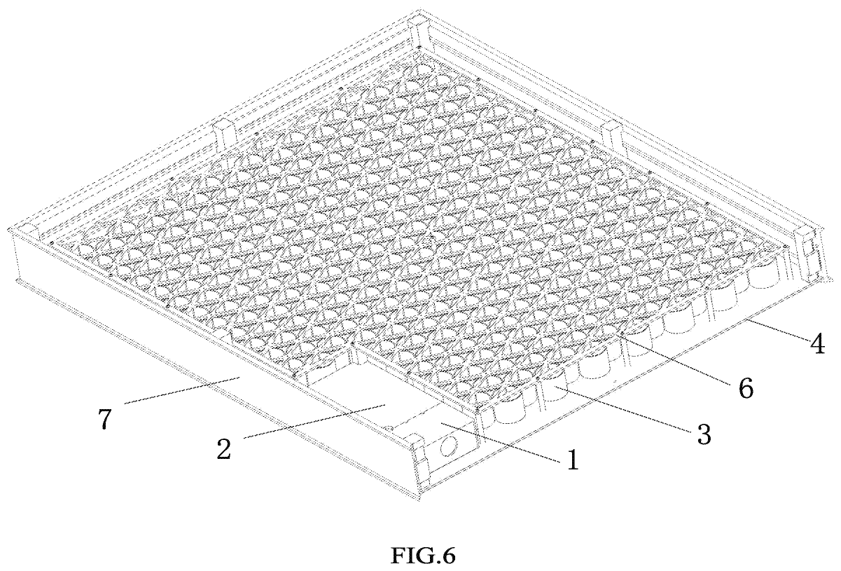

Referring to FIGS. 1-6, the present application provides an ultra-thin corona sterilization module including: an insulation enclosure 7, a apertured negative electrode plate 4 and a positive electrode holder 6; the apertured negative electrode plate 4 is positioned at a bottom of the insulation enclosure 7, and the apertured negative electrode plate 4 is provided with a number of mounting holes for mounting a metal cylinder-shaped tube 3, with a bottom air inlet 9 of the metal cylinder-shaped tube 3 fixed in the mounting holes of the apertured negative electrode plate 4, and a top opening of the metal cylinder-shaped tube 3 as an air outlet 10.

The positive electrode holder 6 is grid-shaped, and the positive electrode holder 6 is positioned on a top of the insulation enclosure 7 and spaced apart from the air outlet 10 of the metal cylinder-shaped tube 3.

There is an electrode needle 5 suspended directly above an axis center of each metal cylinder-shaped tube 3, with an end of the electrode needle 5 mounted at a skeleton of the positive electrode holder 6, with a spacing (5-10 mm is appropriate) between a tip of the electrode needle 5 and the air inlet 9 of the metal cylinder-shaped tube 3.

with the above configuration, the tip of the electrode needle 5 can be located inside the metal cylinder-shaped tube 3, but still apart from a tube mouth (air inlet 9) with a safe distance, which can achieve an uniform discharge of the tip of the needle, and prevent the generation of ozone, forming an effective corona layer 11 between the needle tip of the electrode needle and walls of the tube, that is: an area from the air inlet 9 to a position 5 mm above the tip of the electrode needle become a corona zone, while the area from the position 5 mm above the tip of the electrode needle to the air outlet 10 is an adsorption area 12.

In specific embodiments, a high-speed rotating corona field 11 is formed between the electrode needle 5 (for example, a discharge alloy needle) and the metal cylinder-shaped tube 3, releasing a large number of positive ions and free radicals to achieve strong mineralized sterilization of the viruses passing through the module, while the large number of positive ions would be enriched on particles and adsorbed by the adsorption zone 12 under Coulomb forces, thus achieving air sterilization in a manned environment.

It should also be noted that the electrode needle 5 must be kept strictly in an axis-center position of the metal cylinder-shaped tube 3, which facilitates controlling the uniform discharge of the needle tip, preventing the ozone generation, and ensuring the corona field effect.

In some embodiments, the apertured negative electrode plate 4 and positive electrode holder 6 are fixedly connected to the insulation enclosure 7 via insulating connecting posts 8, respectively.

In some embodiments, the apertured negative electrode plate 4 is configured parallelly to the positive electrode holder 6.

In some embodiments, the positive electrode holder 6 is spaced more than 10 mm from the cylinder-shaped tube 3 to achieve air insulation.

The insulation enclosure 7 is also provided with an external power connection case 1 and a high voltage pack 2, and the external power connection case 1 is electrically connected with the high voltage pack 2. The high voltage pack 2 has a positive pole electrically connected to the positive electrode holder 6, while the negative pole of the high voltage pack 2 is electrically connected to the apertured negative electrode plate 4. In some embodiments, the high voltage pack has a voltage of 4000v-8000v, and a current is 200 μA-1 mA, with a current limit achieved at 1 mA to ensure the safety of human touch.

In the above embodiments, the cylinder-shaped tube 3 has a height of 1 cm-3.6 cm. The present example creatively focuses on the sterilization ability, weakens the dust removal ability, and strictly controls the height of the cylinder-shaped tube 3 between 1-3.6 cm, so that the overall thickness of the product can be controlled between 2-6 cm, thus achieving an ultra-thin design.

The ultra-thin design of the present disclosure is obtained because that the present disclosure focuses a function on air sterilization ability, and does not require a high adsorption ability. Overly long metal cylinder-shaped tube and electrode needle cannot effectively increase the dust removal ability, but the overly long metal cylinder-shaped tube will easily cause electric field breakdown after dust removal and lead to ozone exceeding, and also increase the difficulty of maintenance. More importantly, an overly long tube length will lead to an increased overall thickness of the equipment, which is not beneficial to usage in practical environment. Moreover, in some key epidemic prevention areas, it is needed to achieve a sterilization rate of 90% at one time, which may require 2 layers of equipment to achieve, so the ultra-thin design becomes particularly important.

Therefore, the ultra-thin design of the present disclosure facilitates the sterilization equipment (the overall thickness of the equipment is as thin as 2.5 cm) being adaptable to air conditioners in any style and has a more aesthetically pleasing appearance, and can be the sterilization equipment with more convenient usage. It can also be designed by a combination of multi-layer of the present design to form a combined equipment with enhanced sterilization ability.

The disclosure has been described by using specific examples above, which are merely for the purpose of facilitating understanding of the disclosure and are not intended to limit the disclosure. For a person of ordinary skill in the art, simple deductions, deformations or changes may be made to the above specific implementations according to the idea of the disclosure.

What is claimed is:

1. A corona sterilization module, comprising: an insulation enclosure, an apertured negative electrode plate and a positive electrode holder; wherein,
the apertured negative electrode plate is positioned at a bottom of the insulation enclosure, the apertured negative electrode plate comprising a number of mounting holes for mounting a metal cylinder-shaped tube, the metal cylinder-shaped tube having a bottom air inlet fixed in the mounting holes of the apertured negative electrode plate, and a top opening of the metal cylinder-shaped tube being configured to be an air outlet;
the positive electrode holder is grid-shaped, the positive electrode holder is positioned on a top of the insulated enclosure and spaced apart from the air outlet of the cylinder-shaped tube, with an electrode needle suspended on above of each cylinder-shaped tube, an end of the electrode needle being mounted at a skeleton of the positive electrode holder, and the electrode needle has a needle tip spaced apart from the air inlet of the cylinder-shaped tube; and
an external power connection case and a high voltage pack are provided in the insulation enclosure, the high voltage pack having a positive pole electrically connected to the positive electrode holder and a negative pole electrically connected to the apertured negative electrode plate.

2. The corona sterilization module of claim 1, wherein, the apertured negative electrode plate and the positive electrode holder are fixedly connected to the insulation enclosure via insulated connecting posts, respectively.

3. The corona sterilization module of claim 1, wherein, the apertured negative electrode plate is configured parallelly to the positive electrode holder.

4. The corona sterilization module of claim 1, wherein, the needle tip of the electrode needle is spaced 5 mm-10 mm from the air inlet of the metal cylinder-shaped tube.

5. The corona sterilization module of claim 1, wherein, the positive electrode holder is spaced more than 10 mm from the metal cylinder-shaped tube.

6. The corona sterilization module of claim 1, wherein, the high voltage pack has a voltage of 4000v-8000v and a current of 200 μA-1 mA.

7. The corona sterilization module of claim 1, wherein, the metal cylinder-shaped tube has a height of 1 cm-3.6 cm.

8. The corona sterilization module of claim 1, wherein, the electrode needle is located in an axis-center position inside the metal cylinder-shaped tube.

9. The corona sterilization module of claim 1, wherein, a lower area from the air inlet to a position 5 mm above the needle tip of the electrode needle is configured to be a corona zone and an upper area from the position 5 mm above the needle tip of the electrode needle to the air outlet is configured to be an adsorption zone.

10. The corona sterilization module of claim 2, wherein, a lower area from the air inlet to a position 5 mm above the needle tip of the electrode needle is configured to be a corona zone and an upper area from the position 5 mm above the needle tip of the electrode needle to the air outlet is configured to be an adsorption zone.

11. The corona sterilization module of claim 3, wherein, a lower area from the air inlet to a position 5 mm above the needle tip of the electrode needle is configured to be a corona zone and an upper area from the position 5 mm above the needle tip of the electrode needle to the air outlet is configured to be an adsorption zone.

12. The corona sterilization module of claim 4, wherein, a lower area from the air inlet to a position 5 mm above the needle tip of the electrode needle is configured to be a corona zone and an upper area from the position 5 mm above the needle tip of the electrode needle to the air outlet is configured to be an adsorption zone.

13. The corona sterilization module of claim 5, wherein, a lower area from the air inlet to a position 5 mm above the needle tip of the electrode needle is configured to be a corona zone and an upper area from the position 5 mm above the needle tip of the electrode needle to the air outlet is configured to be an adsorption zone.

14. The corona sterilization module of claim 6, wherein, a lower area from the air inlet to a position 5 mm above the needle tip of the electrode needle is configured to be a corona zone and an upper area from the position 5 mm above the needle tip of the electrode needle to the air outlet is configured to be an adsorption zone.

15. The corona sterilization module of claim 7, wherein, a lower area from the air inlet to a position 5 mm above the needle tip of the electrode needle is configured to be a corona zone and an upper area from the position 5 mm above the needle tip of the electrode needle to the air outlet is configured to be an adsorption zone.

16. The corona sterilization module of claim 8, wherein, a lower area from the air inlet to a position 5 mm above the needle tip of the electrode needle is configured to be a corona zone and an upper area from the position 5 mm above the needle tip of the electrode needle to the air outlet is configured to be an adsorption zone.

17. The corona sterilization module of claim 9, wherein, the corona zone is configured to be a rotating corona field with a mineralization effect on viruses.
